(19) United States
(12) Patent Application Publication
(10) Pub. No.: US 2021/0388037 A1
(43) Pub. Date: Dec. 16, 2021

(54) LEADER SEQUENCE FOR YEAST

(71) Applicant: BASF SE, Ludwigshafen am Rhein (DE)

(72) Inventors: Jingping Zhong, San Diego, CA (US); Xuqiu Tan, San Diego, CA (US); Melissa Ann Scranton, San Diego, CA (US)

(21) Appl. No.: 17/293,240

(22) PCT Filed: Nov. 18, 2019
(86) PCT No.: PCT/US2019/061930

§ 371 Date: May 12, 2021

Related U.S. Application Data
(60) Provisional application No. 62/769,242, filed on Nov. 19, 2018.

(51) Int. Cl. C07K 14/39; C12N 15/81; C12N 1/16; C12N 9/20; C12N 9/24; C12N 9/30

(52) U.S. Cl. CPC C07K 14/39; C12N 15/815; C12N 1/165; C12N 9/20; C12Y 301/01; C12N 2810/70; C12Y 302/01008; C12N 9/242; C12Y 302/01001; C12N 2800/102; C12N 9/2482

(57) ABSTRACT

The present invention relates to a leader peptide which promotes the secretion of recombinant proteins and a nucleic acid sequence encoding the leader peptide as well as expression cassettes, vectors and host cells comprising this leader sequence. Also disclosed is a method for producing a protein using this leader peptide.

### CROSS-REFERENCE TO RELATED APPLICATIONS

This application claims the benefit of priority to U.S. Application No. 62/769,242, filed on Nov. 19, 2018, the contents of which are incorporated herein by reference in their entirety.

### SEQUENCE LISTING

This application includes a nucleotide and amino acid sequence listing in computer readable form (CRF) as an ASC II text (.txt) file according to “Standard for the Presentation of Nucleotide and Amino Acid Sequence Listings in International Patent Applications Under the Patent Cooperation Treaty (PCT)” ST.25. The sequence listing is identified below and is hereby incorporated by reference into the specification of this application in its entirety and for all purposes.

| File Name | Date of Creation | Size (bytes) |
|---|---|---|
| 180263US01_SequenceListing.txt | Nov. 16, 2018 | 32 KB(32,847 bytes) |

### FIELD OF THE INVENTION

The present invention relates to a leader peptide which promotes the secretion of recombinant proteins from a host cell and a nucleic acid sequence encoding the leader peptide as well as expression cassettes, vectors and host cells comprising this leader sequence. Also disclosed is a method for producing a protein using this leader peptide.

### BACKGROUND OF THE INVENTION

Komagataella phaffii (formerly designated as Pichia pastoris) is a single-celled microorganism that is easy to manipulate and culture. K. phaffii is a eukaryote capable of many of the post-translational modifications performed by higher eukaryotic cells such as proteolytic processing, folding, disulfide bond formation and glycosylation. Thus, the K. phaffii system is preferred over bacterial systems which are not capable of performing the same post-translation modifications as eukaryotic cells. Further, in bacterial systems proteins may be produced in insoluble form which requires expensive processes to refold and recover the proteins, if possible at all. Additionally, the K. phaffii system has been shown to give more soluble and relatively pure secreted protein than many bacterial systems. Hence, foreign proteins requiring post-translational modifications may be produced as biologically active molecules in K. phaffii and K. phaffii is already used for the production of a wide variety of recombinant proteins.

As the majority of yeasts do not secrete large amounts of endogenous proteins, and their extracellular proteomes are not extensively characterized so far, the number of available secretion sequences for use in yeasts is limited. Therefore, the target protein is typically fused to the leader peptide of mating factor alfa (MFa) from S. cerevisiae to drive secretory expression in many yeast species (Kurjan and Herskowitz (1982) Cell 30(3): 933-943). However, the proteolytic processing of the MFa by Kex2 protease often yields heterogeneous N-terminal amino acid residues in the product.

EP 0 324 274 B1 describes improved expression and secretion of heterologous proteins in yeast using truncated S. cerevisiae alfa-factor leader sequences.

The genome sequencing of Pichia pastoris led to the identification of 54 putative signal peptides (De Schutter et al. (2009) Nature Biotechnol. 27(6): 561-566 and supplementary information).

WO 2014/067926 A1 discloses protein expression and secretion using a mutated Epx1 leader peptide.

Nevertheless, there is still a need for leader peptides which effect the high level secretion of recombinant proteins from yeast cells.

### SUMMARY OF THE INVENTION

The present inventors have isolated a leader peptide which provides for strong expression and secretion of a protein associated therewith and can therefore be used in the production of recombinant proteins.

Accordingly, the present invention relates to an isolated leader peptide selected from the group consisting of:

(a) a peptide comprising the amino acid sequence according to SEQ ID No. 1 or a functional variant thereof;
(b) a peptide comprising an amino acid sequence selected from the group of SEQ ID No. 2, SEQ ID No. 3, SEQ ID No. 4 and SEQ ID No. 5, or a functional variant thereof; and
(c) a peptide comprising an amino acid sequence which is at least 80% identical to the amino acid sequence according to any one of SEQ ID Nos. 1, SEQ ID No. 2, SEQ ID No. 3, SEQ ID No. 4 and SEQ ID No. 5.

The present invention further relates to an isolated nucleic acid molecule comprising a nucleic acid sequence which encodes a leader peptide according to claim 1.

In one embodiment, the nucleic acid sequence is selected from the group consisting of:

(a) a nucleic acid sequence encoding a peptide comprising an amino acid sequence according to any one of SEQ ID Nos. 1, SEQ ID No. 2, SEQ ID No. 3, SEQ ID No. 4 and SEQ ID No. 5;
(b) a nucleic acid sequence comprising the sequence according to any one of SEQ ID Nos. 6, SEQ ID No. 7, SEQ ID No. 8, SEQ ID No. 9 and SEQ ID No. 10;
(c) a nucleic acid sequence which is at least 80% identical to the nucleic acid sequence according to any one of SEQ ID Nos. 6, SEQ ID No. 7, SEQ ID No. 8, SEQ ID No. 9 and SEQ ID No. 10; and
(d) a nucleic acid sequence hybridizing under stringent conditions to the nucleic acid sequence according to any one of SEQ ID Nos. 6, SEQ ID No. 7, SEQ ID No. 8, SEQ ID No. 9 and SEQ ID No. 10.

The present invention further relates to an expression cassette comprising the nucleic acid molecule of the present invention operably linked to a nucleic acid sequence encoding a protein.

The protein may be an enzyme, a peptide, an antibody or antigen-binding fragment thereof, a protein antibiotic, a fusion protein, a vaccine or a vaccine-like protein or particle, a growth factor, a hormone or a cytokine.

The enzyme may be selected from the group consisting of lipase, amylase, glucoamylase, protease, xylanase, glucanase, cellulase, mannanase and phytase.

The expression cassette may further comprise a promoter operably linked to said nucleic acid molecule.

The present invention further relates to a vector comprising the expression cassette of the present invention and to a host cell comprising the expression cassette of the present invention or the vector of the present invention.

The host cell may be a yeast cell which may be selected from the group consisting of Komagataella, Candida, Torulopsis, Arxula, Hansenula, Ogatea, Yarrowia, Kluyveromyces, Ashbya and Saccharomyces.

The present invention further relates to a method for producing a protein in a host cell, comprising the steps of:

(a) providing a host cell of the present invention;
(b) culturing the host cell under suitable conditions; and
(c) obtaining the protein.

The present invention further relates to the use of the nucleic acid sequence of the present invention or the leader peptide of the present invention for the secretion of a protein from a host cell and/or for increasing the secretion of a protein from a host cell.

### BRIEF DESCRIPTION OF THE DRAWINGS

FIGS. 1A-1B: Expression of lipase A fused either to the alpha-factor leader peptide or the leader peptide according to SEQ ID No. 2 called AmyTZ (a) or the leader peptide according to SEQ ID No. 3 called Nectria (b).

FIG. 2: Expression of xylanase fused either to the alpha-factor leader peptide, the leader peptide according to SEQ ID No. 2 called AmyTZ or the native signal peptide. Numbers 1˜4 represent different colonies.

FIG. 3: Expression of amylase fused either to the alpha-factor leader peptide (right figure) or the leader peptide according to SEQ ID No. 2 called AmyTZ (left figure). Each lane represents individual transformants.

FIGS. 4A-4B: Expression (a) and activity (b) of lipase B fused either to the alpha-factor leader peptide or the leader peptide according to SEQ ID No. 2 called AmyTZ.

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The term “isolated nucleic acid molecule” refers to a nucleic acid molecule that has been separated from the environment with which it is naturally associated, such as the genome. In the context of the leader peptide disclosed herein the term particularly means that the isolated nucleic acid molecule encoding the leader peptide has been separated from the nucleic acid molecule encoding the protein to which the leader peptide is naturally linked.

The terms “nucleic acid”, “nucleic acid sequence” or “nucleic acid molecule” have their usual meaning and may include, but are not limited to, for example, polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acids can be either single-stranded or double-stranded. In some embodiments, a nucleic acid sequence encoding a fusion protein or recombinant protein is provided, wherein the protein is linked to the leader peptide of the present invention.

The nucleic acid sequences of the present invention further encompass codon-optimized sequences, which encode the leader peptide of the present invention. A nucleic acid is codon-optimized by systematically altering codons in recombinant DNA to be expressed in a host cell other than the cell from which the nucleic acid was isolated so that the codons match the pattern of codon usage in the organism used for expression and thereby to enhance yields of an expressed protein. The codon-optimized sequence nevertheless encodes a protein with the same amino acid sequence as the native protein.

The terms “coding for” or “encoding” as used herein have their usual meaning and may include, but are not limited to, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. In some embodiments of the present invention a nucleic acid sequence encoding a protein is used, wherein the nucleic acid sequence encoding the protein is operably linked to a nucleic acid sequence encoding the leader peptide of the present invention.

The term “leader peptide” as used herein refers to a peptide which directs the secretion of a protein. Proteins which are secreted from a cell have a leader peptide located at the N-terminus of the protein which is cleaved from the mature protein once the export of the nascent protein chain across the rough endoplasmic reticulum has been initiated. A leader peptide enables an expressed protein to be transported to or across the plasma membrane, thereby making it easy to separate and purify the expressed protein. Usually, leader peptides are cleaved from the protein by specialized cellular peptidases after the proteins have been transported to or across the plasma membrane.

The term “functional variant” as used herein with respect to the leader peptide of the present invention is intended to refer to those variants with one or two point mutations in the amino acid sequence, which have essentially the same leader activity as compared to the unmodified sequences. Hence, a functional variant of the peptide according to SEQ ID No. 1 has one or two amino acid exchanges compared to SEQ ID No. 1 and substantially the same leader activity as the unmodified peptide according to SEQ ID No. 1. A functional variant of the peptide according to any one of SEQ ID Nos. 2 to 5 has one or two amino acid exchanges compared to the corresponding sequence of any one of SEQ ID Nos. 2 to 5 and substantially the same leader activity as the corresponding unmodified peptide according to any one of SEQ ID Nos. 2 to 5.

A functional variant of the leader peptide of the present invention has essentially the same leader activity as the unmodified sequence, if the fusion of the variant leader peptide to a protein leads to essentially the same secretion of said protein into the supernatant by the recombinant host cell as the fusion of the unmodified leader sequence to said protein. Essentially the same secretion means that the amount of the protein in the supernatant of a host cell expressing the functional variant of the leader peptide is at least 50% or 60%, preferably at least 70% or 75%, more preferably at least 80% or 85% and most preferably at least 90%, 92%, 95% or 98% of the amount of the protein in the supernatant of the host cell expressing the unmodified leader peptide.

“Sequence Identity”, “% sequence identity”, “% identity”, “% identical” or “sequence alignment” means a comparison of a first amino acid sequence to a second amino acid sequence, or a comparison of a first nucleic acid sequence to a second nucleic acid sequence and is calculated as a percentage based on the comparison. The result of this calculation can be described as “percent identical” or “percent ID.”

Generally, a sequence alignment can be used to calculate the sequence identity by one of two different approaches. In the first approach, both mismatches at a single position and gaps at a single position are counted as non-identical positions in final sequence identity calculation. In the second approach, mismatches at a single position are counted as nonidentical positions in final sequence identity calculation; however, gaps at a single position are not counted (ignored) as non-identical positions in final sequence identity calculation. In other words, in the second approach gaps are ignored in final sequence identity calculation. The difference between these two approaches, i.e. counting gaps as non-identical positions vs ignoring gaps, at a single position can lead to variability in the sequence identity value between two sequences.

A sequence identity is determined by a program, which produces an alignment, and calculates identity counting both mismatches at a single position and gaps at a single position as non-identical positions in final sequence identity calculation. For example program Needle (EMBOS), which has implemented the algorithm of Needleman and Wunsch (Needleman and Wunsch, 1970, J. Mol. Biol. 48: 443-453), and which calculates sequence identity per default settings by first producing an alignment between a first sequence and a second sequence, then counting the number of identical positions over the length of the alignment, then dividing the number of identical residues by the length of an alignment, then multiplying this number by 100 to generate the % sequence identity [% sequence identity=(# of Identical residues/length of alignment)×100)].

A sequence identity can be calculated from a pairwise alignment showing both sequences over the full length, so showing the first sequence and the second sequence in their full length (“Global sequence identity”). For example, program Needle (EMBOSS) produces such alignments; % sequence identity=(# of identical residues/length of alignment)×100)].

A sequence identity can be calculated from a pairwise alignment showing only a local region of the first sequence or the second sequence (“Local Identity”). For example, program Blast (NCBI) produces such alignments; % sequence identity=(# of Identical residues/length of alignment)×100)].

The sequence alignment is preferably generated by using the algorithm of Needleman and Wunsch (J. Mol. Biol. (1979) 48, p. 443-453). Preferably, the program “NEEDLE” (The European Molecular Biology Open Software Suite (EMBOSS)) is used with the programs default parameter (gap open=10.0, gap extend=0.5 and matrix=EBLOSUM62 for proteins and matrix=EDNAFULL for nucleotides). Then, a sequence identity can be calculated from the alignment showing both sequences over the full length, so showing the first sequence and the second sequence in their full length (“Global sequence identity”). For example: % sequence identity=(# of identical residues/length of alignment)×100)].

The variant nucleic acid sequences are described by reference to a nucleic acid sequence which is at least n % identical to the nucleic acid sequence of the respective parent peptide with “n” being an integer between 80 and 100. The variant nucleic acid sequences include sequences that are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical when compared to the full-length sequence of the parent nucleic acid according to any one of SEQ ID Nos. 6-10, wherein the variant nucleic acid encodes a peptide having essentially the same leader activity as the parent peptide.

The variant peptides are described by reference to an amino acid sequence which is at least n % identical to the amino acid sequence of the respective parent peptide with “n” being an integer between 80 and 100. The variant peptides include sequences that are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical when compared to the full-length sequence of the parent peptide according to any one SEQ ID Nos. 1-5, wherein the variant peptide has essentially the same leader activity as the parent peptide.

The nucleic acid sequence hybridizing under stringent conditions with a complementary sequence of a nucleic acid sequence selected from the group consisting of SEQ ID No. 1, SEQ ID No. 2, SEQ ID No. 3, SEQ ID No. 4 and SEQ ID No. 5 encodes a peptide having essentially the same leader activity as the parent peptide according to any one of SEQ ID Nos. 1-5.

The term “hybridizing under stringent conditions” denotes in the context of the present invention that the hybridization is implemented in vitro under conditions which are stringent enough to ensure a specific hybridization. Stringent in vitro hybridization conditions are known to those skilled in the art and may be taken from the literature (e.g. Sambrook and Russell (2001) Molecular Cloning: A Laboratory Manual, 3rd edition, Cold Spring Harbour Laboratory Press, Cold Spring Harbour, N.Y.). The term “specific hybridization” refers to the circumstance that a molecule, under stringent conditions, preferably binds to a certain nucleic acid sequence, i.e. the target sequence, if the same is part of a complex mixture of, e.g. DNA or RNA molecules, but does not, or at least very rarely, bind to other sequences.

Stringent conditions depend on the circumstances. Longer sequences hybridize specifically at higher temperatures. In general, stringent conditions are chosen such that the hybridization temperature is about 5° C. below the melting point (Tₘ) of the specific sequence at a defined ionic strength and at a defined pH value. Tₘ is the temperature (at a defined pH value, a defined ionic strength and a defined nucleic acid concentration), at which 50% of the molecules complementary to the target sequence hybridize to the target sequence in the state of equilibrium. Typically, stringent conditions are conditions, where the salt concentration has a sodium ion concentration (or concentration of a different salt) of at least about 0.01 to 1.0 M at a pH value between 7.0 and 8.3, and the temperature is at least 30° C. for small molecules (i.e. 10 to 50 nucleotides, for example). In addition, stringent conditions may include the addition of substances, such as, e. g., formamide, which destabilise the hybrids. At hybridization under stringent conditions, as used herein, normally nucleotide sequences which are at least 60% homologous to each other hybridize to each other. Preferably, said stringent conditions are chosen such that sequences which are about 65%, preferably at least about 70%, and especially preferably at least about 75% or higher homologous to each other, normally remain hybridized to each other. A preferred but non-limiting example of stringent hybridization conditions is hybridizations in 6×sodium chloride/sodium citrate (SSC) at about 45° C., followed by one or more washing steps in 0.2×SSC, 0.1% SDS at 50 to 65° C. The temperature depends on the type of the nucleic acid and is between 42° C. and 58° C. in an aqueous buffer having a concentration of 0.1 to 5×SSC (pH value 7.2).

If an organic solvent, e.g. 50% formamide, is present in the above-mentioned buffer, the temperature is about 42° C. under standard conditions. Preferably, the hybridisation conditions for DNA:DNA hybrids are, for example, 0.1×SSC and 20° C. to 45° C., preferably 30° C. to 45° C. Preferably, the hybridisation conditions for DNA:RNA hybrids are, for example, 0.1×SSC and 30° C. to 55° C., preferably between 45° C. and 55° C. The above-mentioned hybridization temperatures are determined, for example, for a nucleic acid which is 100 base pairs long and has a G/C content of 50% in the absence of formamide. Those skilled in the art know how to determine the required hybridization conditions using text books such as those mentioned above or the following textbooks: Current Protocols in Molecular Biology, John Wiley & Sons, N.Y. (1989), Hames and Higgins (publ.) 1985, Nucleic Acids Hybridization: A Practical Approach, IRL Press at Oxford University Press, Oxford; Brown (publ.) 1991, Essential Molecular Biology: A Practical Approach, IRL Press at Oxford University Press, Oxford.

Typical hybridization and washing buffers for example have the following composition:

Pre-hybridization solution: 0.5% SDS
5×SSC
50 mM sodium phosphate, pH 6.8
0.1% sodium pyrophosphate
5×Denhardt's solution
100 μg/mL salmon sperm DNA

Hybridization solution: pre-hybridization solution
1×10⁶ cpm/mL probe (5-10 min 95° C.)

20×SSC: 3 M NaCl
0.3 M sodium citrate
ad pH 7 with HCl

50×Denhardt's reagent: 5 g Ficoll
5 g polyvinylpyrrolidone
5 g bovine serum albumin
ad 500 mL aqua destillata

A typical procedure for hybridization is as follows:

| Optional: | wash blot 30 min in 1x SSC/0.1% SDS at 65° C. | | |
|---|---|---|---|
| Pre-hybridization: | at least 2 h at 50-55° C. | | |
| Hybridization: | over night at 55-60° C. | | |
| Washing: | 5 min | 2x SSC/0.1% SDS | hybridization temp. |
| | 30 min | 2x SSC/0.1% SDS | hybridization temp. |
| | 30 min | 1x SSC/0.1% SDS | hybridization temp. |
| | 45 min | 0.2x SSC/0.1% SDS | 65° C. |
| | 5 min | 0.1x SSC | room temperature |

Those skilled in the art know that the given solutions and the presented protocol may be modified or have to be modified, depending on the application.

As discussed above, “essentially the same leader activity” means that the fusion of the leader peptide having the above-described sequence identity to the unmodified leader peptide of any one of SEQ ID Nos. 1-5 to a protein leads to essentially the same secretion of said protein into the supernatant by the recombinant host cell as the fusion of the unmodified leader sequence to said protein. Essentially the same secretion means that the amount of the protein in the supernatant of a host cell expressing the leader peptide having the above-described sequence identity to the unmodified leader peptide of any one of SEQ ID Nos. 1-5 is at least 50% or 60%, preferably at least 70% or 75%, more preferably at least 80% or 85% and most preferably at least 90%, 92%, 95% or 98% of the amount of the protein in the supernatant of the host cell expressing the unmodified leader peptide.

The term “expression cassette” refers to a nucleic acid molecule containing the coding sequence of a protein and control sequences such as e.g. a promoter in operable linkage, so that host cells transformed or transfected with these sequences are capable of producing the encoded proteins. The expression cassette may be part of a vector or may be integrated into the host cell chromosome. In the expression cassette of the present invention the nucleic acid sequence encoding the leader peptide of the present invention is operably linked to the nucleic acid sequence encoding the protein so that upon transcription of the nucleic acid sequence and translation the leader peptide and the protein are linked by a peptide bond.

The protein which can be expressed and secreted using the leader peptide of the present invention can be any protein such as any eukaryotic, prokaryotic and synthetic protein. The protein may be homologous to the host cell, i.e. it may be naturally expressed by the host cell, or it can be heterologous to the host cell, i.e. it may not be naturally expressed by the host cell. The protein can include, but is not limited to, enzymes, peptides, antibodies and antigen-binding fragments thereof and recombinant proteins. Proteins obtained by heterologous expression in K. phaffii which are already on the market include phytase, trypsin, nitrate reductase, phospholipase C, collagen, proteinase K, ecallantide, ocriplasmin, human insulin, pleactasin peptide derivative NZ2114, elastase inhibitor, recombinant cytokines and growth factors, human cystatin C, HB-EGF, interferon-alpha 2b, human serum albumin and human angiostatin.

In one embodiment the protein is an enzyme. The enzyme may be selected from the group consisting of lipase, amylase, glucoamylase, protease, xylanase, glucanase, cellulase, mannanase and phytase.

In one embodiment, the protein is a lipase. The lipase may have an amino acid sequence having at least 80% sequence identity to the amino acid sequence of SEQ ID No. 23. In one embodiment, the lipase has an amino acid sequence according to SEQ ID No. 23. In one embodiment, the lipase is encoded by a nucleic acid sequence having at least 80% sequence identity to the nucleic acid sequence of SEQ ID No. 22. In one embodiment, the lipase is encoded by the nucleic acid sequence according to SEQ ID No. 22. The protein having an amino acid sequence which is at least 80% identical to the amino acid sequence of SEQ ID No. 23 or which is encoded by a nucleic acid sequence which is at least 80% identical to the nucleic acid sequence of SEQ ID No. 22 and has lipase activity. The term “lipase activity” means that the protein can cleave ester bonds in lipids. The lipase activity of a protein can be determined by incubating the protein with a suitable lipase substrate, such as PNP-octanoate, 1-olein, galactolipids, phosphatidylcholine and triacylglycerols and determining the lipase activity in comparison to a control lipase.

In one embodiment, the lipase comprises one or more amino acid insertions, deletions or substitutions in comparison to the amino acid sequence of SEQ ID No. 23. In one embodiment, the amino acid insertion, deletion or substitution in comparison to the amino acid sequence of SEQ ID No. 23 is at an amino acid residue selected from amino acid residues 23, 33, 82, 83, 84, 85, 160, 199, 254, 255, 256, 258, 263, 264, 265, 268, 308 and 311. In one embodiment, the amino acid substitution in comparison to the amino acid sequence of SEQ ID No. 23 is selected from the group consisting of: Y23A, K33N, S82T, S83D, S83H, S83I, S83N, S83R, S83T, S83Y, S84S, S84N, I85A, I85C, I85F, I85H, I85L, I85M, I85P, I85S, I85T, I85V, I85Y, K160N, P1991, P199V, I254A, I254C, I254E, I254F, I254G, I254L, I254M, I254N, I254R, I254S, 12454V, I254W, I254Y, I255A, I255L, A256D, L258A, L258D; L258E, L258G, L258H, L258N, L258Q, L258R, L258S, L258T, L258V, D263G, D263K, D263P, D263R, D263S; T264A, T264D, T264G, T2641, T264L, T264N, T2645, D265A, D265G, D265K, D265L, D265N, D2655, D265T, T268A, T268G, T268K, T268L, T268N, T2685, D308A, and Y311E.

Further suitable lipases having one or more amino acid substitutions or insertions compared to the sequence according to SEQ ID No. 23 are shown in the following Table 1 wherein LIP062 refers to the lipase according to SEQ ID No. 23.

TABLE 1

| | Amino Acid Residue Position Numbers | | | | | | | | | | | | | | | | | | |
|---|---|---|---|---|---|---|---|---|---|---|---|---|---|---|---|---|---|---|---|
| Lipase | 23 | 33 | 82 | 83 | 84 | 84′ | 85 | 160 | 199 | 254 | 255 | 256 | 258 | 263 | 264 | 265 | 268 | 308 | 311 |
| LIP062 | Y | K | S | S | N | — | I | K | P | I | I | A | L | D | T | D | T | D | Y |
| LIP182 | — | — | — | H | — | — | S | — | — | — | A | — | — | — | A | T | — | — | — |
| LIP181 | — | — | — | H | — | — | V | — | — | — | A | — | — | — | S | T | — | — | — |
| LIP180 | — | — | — | T | — | — | H | — | — | — | A | — | — | — | — | A | — | — | — |
| LIP179 | — | — | — | — | — | — | V | — | — | — | A | — | — | — | S | T | — | — | — |
| LIP178 | — | — | — | H | — | — | L | — | — | — | A | — | — | — | S | A | — | — | — |
| LIP177 | — | — | — | H | — | — | T | — | — | — | A | — | — | — | — | T | — | — | — |
| LIP176 | — | — | — | Y | — | — | A | — | — | — | A | — | — | — | — | T | — | — | — |
| LIP175 | — | — | — | T | — | — | V | — | — | — | A | — | — | — | — | S | — | — | — |
| LIP174 | — | — | — | — | — | — | — | — | — | — | A | — | — | — | — | A | — | — | — |
| LIP173 | — | — | — | — | — | — | — | — | — | — | A | — | — | — | — | S | — | — | — |
| LIP172 | — | — | — | N | — | — | L | — | — | — | A | — | — | — | N | T | — | — | — |
| LIP171 | — | — | — | — | — | — | — | — | — | — | A | — | — | — | D | T | — | — | — |
| LIP170 | — | — | — | N | — | — | L | — | — | — | A | — | — | — | — | T | — | — | — |
| LIP169 | — | — | — | N | — | — | V | — | — | L | — | — | — | — | S | T | — | — | — |
| LIP168 | — | — | — | H | — | — | — | — | — | L | — | — | — | — | A | A | — | — | — |
| LIP167 | — | — | — | H | — | — | — | — | — | L | — | — | — | — | — | T | — | — | — |
| LIP166 | — | — | — | — | — | — | V | — | — | L | — | — | — | — | D | T | — | — | — |
| LIP165 | — | — | — | Y | — | — | — | — | — | — | — | — | — | — | A | T | — | — | — |
| LIP164 | — | — | — | — | — | — | V | — | — | — | — | — | — | — | D | T | — | — | — |
| LIP163 | — | — | — | Y | — | — | A | — | — | — | — | — | — | — | A | T | — | — | — |
| LIP162 | — | — | — | N | — | — | V | — | — | — | — | — | — | — | N | T | — | — | — |
| LIP161 | — | — | — | N | — | — | — | — | — | — | — | — | — | — | D | T | — | — | — |
| LIP160 | — | — | — | H | — | — | L | — | — | — | — | — | — | — | — | T | — | — | — |
| LIP159 | — | — | — | H | — | — | A | — | — | — | — | — | — | — | A | T | — | — | — |
| LIP158 | — | — | — | T | — | — | V | — | — | — | — | — | — | — | — | T | — | — | — |
| LIP157 | — | — | — | H | — | — | L | — | — | — | — | — | — | — | — | A | — | — | — |
| LIP156 | — | — | — | H | — | — | V | — | — | — | — | — | — | — | — | A | — | — | — |
| LIP155 | — | — | — | T | — | — | A | — | — | — | — | — | — | — | — | T | — | — | — |
| LIP154 | — | — | — | H | — | — | V | — | — | — | — | — | — | — | N | T | — | — | — |
| LIP153 | — | — | — | — | — | — | V | — | — | — | — | — | — | — | — | G | — | — | — |
| LIP152 | — | — | — | H | — | — | — | — | — | — | — | — | — | — | — | A | — | — | — |
| LIP151 | — | — | — | Y | — | — | V | — | — | — | — | — | — | — | S | S | — | — | — |
| LIP150 | — | — | — | N | — | — | V | — | — | — | — | — | — | — | — | G | — | — | — |
| LIP149 | — | — | — | H | — | — | — | — | — | — | — | — | — | — | — | S | — | — | — |
| LIP148 | — | — | — | H | — | — | — | — | — | — | — | — | — | — | — | G | — | — | — |
| LIP147 | — | — | — | H | — | — | — | — | — | — | — | — | — | — | — | S | G | — | — |
| LIP146 | — | — | — | H | — | — | — | — | — | — | — | — | — | — | — | G | G | — | — |
| LIP145 | — | — | — | — | — | — | — | — | — | — | A | — | — | — | — | S | G | — | — |
| LIP144 | — | — | — | H | — | — | — | — | — | — | A | — | — | — | — | G | — | — | — |
| LIP143 | — | — | — | H | — | — | — | — | — | — | A | — | — | — | — | S | G | — | — |
| LIP142 | — | — | — | H | — | — | — | — | — | — | A | — | — | — | — | G | G | — | — |
| LIP135 | — | — | — | — | — | — | — | — | — | — | L | — | — | — | — | — | — | — | — |
| LIP134 | — | — | — | — | — | — | — | — | — | — | A | — | — | — | — | — | — | — | — |
| LIP131 | — | — | — | I | — | — | L | — | — | — | — | — | — | — | — | S | — | — | — |
| LIP130 | — | — | — | I | — | — | L | — | — | — | — | — | — | — | — | G | — | — | — |
| LIP126 | — | — | — | — | — | — | — | — | — | — | — | — | — | R | — | — | — | — | — |
| LIP124 | — | — | — | — | — | — | T | — | — | — | — | — | — | — | — | G | G | — | — |
| LIP123 | — | — | — | — | — | — | L | — | — | — | — | — | — | — | — | G | G | — | — |
| LIP120 | — | — | — | H | — | — | L | — | — | — | — | — | — | — | — | — | G | — | — |
| LIP119 | — | — | — | — | — | — | T | — | — | — | — | — | — | — | — | S | G | — | — |
| LIP118 | — | — | — | H | — | — | L | — | — | — | — | — | — | — | — | G | — | — | — |
| LIP117 | — | — | — | H | — | — | T | — | — | — | — | — | — | — | — | S | G | — | — |
| LIP116 | — | — | — | H | — | — | L | — | — | — | — | — | — | — | — | G | G | — | — |
| LIP115 | — | — | — | H | — | — | L | — | — | — | — | — | — | — | — | S | G | — | — |
| LIP114 | — | — | — | H | — | — | L | — | — | — | — | — | — | — | — | S | — | — | — |
| LIP113 | — | — | — | — | — | — | L | — | — | — | — | — | — | — | — | S | G | — | — |
| LIP111 | — | — | — | — | — | — | — | — | — | — | — | — | — | — | — | A | — | — | — |
| LIP110 | — | — | — | — | — | — | — | — | — | — | — | — | — | — | — | S | — | — | — |
| LIP109 | — | — | — | — | — | — | — | — | — | — | — | — | — | — | — | G | — | — | — |
| LIP108 | — | — | — | H | — | — | — | — | — | — | — | — | — | — | — | — | — | — | — |
| LIP102 | — | — | — | — | — | — | T | — | — | — | — | — | — | — | — | — | — | — | — |
| LIP101 | — | — | — | — | — | — | P | — | — | — | — | — | — | — | — | — | — | — | — |
| LIP100 | — | — | — | — | — | — | L | — | — | — | — | — | — | — | — | — | — | — | — |
| LIP099 | — | — | — | — | — | — | A | — | — | — | — | — | — | — | — | — | — | — | — |
| LIP096 | — | — | — | — | — | — | — | — | — | — | — | D | — | — | — | — | — | — | — |
| LIP095 | — | — | — | — | — | — | — | N | — | — | — | — | — | — | — | — | — | — | — |
| LIP094 | — | N | — | — | — | — | — | — | — | — | — | — | — | — | — | — | — | — | — |
| LIP090 | — | — | — | — | — | — | — | — | V | — | — | — | — | — | — | — | — | — | — |
| LIP089 | — | — | T | — | — | — | — | — | — | — | — | — | — | — | — | — | — | — | — |
| LIP062_1909 | — | — | — | — | — | — | T | — | — | — | A | — | — | — | — | — | — | — | — |
| LIP062_1908 | — | — | — | H | — | — | T | — | — | — | A | — | — | — | — | — | — | — | — |
| LIP062_1907 | — | — | — | — | — | — | P | — | — | — | A | — | — | — | — | S | — | — | — |
| LIP062_1906 | — | — | — | H | — | — | P | — | — | — | A | — | — | — | — | — | — | — | — |
| LIP062_1905 | — | — | — | I | — | — | — | — | — | — | A | — | — | — | — | G | G | — | — |
| LIP062_1904 | — | — | — | — | — | — | — | — | — | — | A | — | — | — | — | G | G | — | — |
| LIP062_1903 | — | — | — | H | — | — | P | — | — | — | — | — | — | — | — | — | G | — | — |
| LIP062_1902 | — | — | — | — | — | — | P | — | — | — | — | — | — | — | — | S | G | — | — |
| LIP062_1901 | — | — | — | — | — | — | T | — | — | — | — | — | — | — | — | S | — | — | — |
| LIP062_1900 | — | — | — | H | — | — | T | — | — | — | — | — | — | — | — | — | — | — | — |
| LIP062_1899 | — | — | — | — | — | — | P | — | — | — | — | — | — | — | — | S | — | — | — |
| LIP062_1898 | — | — | — | H | — | — | P | — | — | — | — | — | — | — | — | — | — | — | — |
| LIP062_1897 | — | — | — | I | — | — | — | — | — | — | — | — | — | — | — | G | — | — | — |
| LIP062_1896 | — | — | — | — | — | — | — | — | — | — | — | — | — | — | — | S | G | — | — |
| LIP062_1895 | — | — | — | I | — | — | — | — | — | — | — | — | — | — | — | G | G | — | — |
| LIP062_1894 | — | — | — | — | — | — | — | — | — | — | — | — | — | — | — | G | G | — | — |
| LIP062_1893 | — | — | — | I | — | — | T | — | — | — | — | — | — | — | — | — | G | — | — |
| LIP062_1892 | — | — | — | H | — | — | T | — | — | — | — | — | — | — | — | — | G | — | — |
| LIP062_1891 | — | — | — | I | — | — | T | — | — | — | — | — | — | — | — | S | — | — | — |
| LIP062_1890 | — | — | — | I | — | — | T | — | — | — | — | — | — | — | — | G | — | — | — |
| LIP062_1889 | — | — | — | H | — | — | T | — | — | — | — | — | — | — | — | S | — | — | — |
| LIP062_1888 | — | — | — | H | — | — | T | — | — | — | — | — | — | — | — | G | — | — | — |
| LIP062_1887 | — | — | — | I | — | — | L | — | — | — | — | — | — | — | — | — | G | — | — |
| LIP062_1886 | — | — | — | I | — | — | T | — | — | — | — | — | — | — | — | S | G | — | — |
| LIP062_1885 | — | — | — | I | — | — | L | — | — | — | — | — | — | — | — | S | G | — | — |
| LIP062_1884 | — | — | — | I | — | — | T | — | — | — | — | — | — | — | — | G | G | — | — |
| LIP062_1883 | — | — | — | I | — | — | L | — | — | — | — | — | — | — | — | G | G | — | — |
| LIP062_1882 | — | — | — | H | — | — | T | — | — | — | — | — | — | — | — | G | G | — | — |
| LIP062_1881 | — | — | — | — | — | — | — | — | — | — | — | — | — | — | I | — | — | — | — |
| LIP062_1880 | — | — | — | — | — | — | — | — | — | — | — | — | — | — | L | — | — | — | — |
| LIP062_1879 | — | — | — | — | — | — | — | — | — | — | — | — | — | P | — | — | — | — | — |
| LIP062_1878 | — | — | — | — | — | — | — | — | — | — | — | — | — | G | — | — | — | — | — |
| LIP062_1877 | — | — | — | — | — | — | — | — | — | — | — | — | — | S | — | — | — | — | — |
| LIP062_1876 | — | — | — | — | — | — | — | — | — | — | — | — | — | K | — | — | — | — | — |
| LIP062_1875 | — | — | — | I | N | — | V | — | — | — | — | — | — | — | — | — | — | — | — |
| LIP062_1874 | — | — | — | R | S | — | V | — | — | — | — | — | — | — | — | — | — | — | — |
| LIP062_1873 | — | — | — | — | — | — | — | — | — | — | — | — | — | — | — | — | L | — | — |
| LIP062_1872 | — | — | — | — | — | — | — | — | — | — | — | — | — | — | — | — | A | — | — |
| LIP062_1871 | — | — | — | — | — | — | — | — | — | — | — | — | — | — | — | — | N | — | — |
| LIP062_1870 | — | — | — | — | — | — | — | — | — | — | — | — | — | — | — | — | K | — | — |
| LIP062_1869 | — | — | — | — | — | — | — | — | — | — | — | — | — | — | — | — | S | — | — |
| LIP062_1868 | — | — | — | — | — | — | — | — | — | — | — | — | — | — | — | — | G | — | — |
| LIP062_1867 | — | — | — | — | — | — | — | — | — | — | — | — | — | — | — | L | — | — | — |
| LIP062_1866 | — | — | — | — | — | — | — | — | — | — | — | — | — | — | — | N | — | — | — |
| LIP062_1865 | — | — | — | — | — | — | — | — | — | — | — | — | — | — | — | K | — | — | — |
| LIP062_1864 | — | — | — | N | — | — | — | — | — | — | — | — | — | — | — | — | — | — | — |
| LIP062_1863 | — | — | — | D | — | — | — | — | — | — | — | — | — | — | — | — | — | — | — |
| LIP062_1862 | — | — | — | I | — | — | — | — | — | — | — | — | — | — | — | — | — | — | — |
| LIP062_1861 | A | — | — | — | — | — | — | — | — | — | — | — | — | — | — | — | — | — | — |
| LIP062_1860 | — | — | — | — | — | — | — | — | — | — | — | — | — | — | — | — | — | A | E |
| LIP062_1859 | — | — | — | — | — | — | — | — | — | — | — | — | — | — | — | — | — | A | — |
| LIP062_1858 | — | — | — | — | — | — | — | — | — | — | — | — | — | — | — | — | — | — | E |
| LIP062_1857 | — | — | — | — | — | S | — | — | — | — | — | — | — | — | — | — | — | — | — |
| LIP062_1856 | — | — | — | — | — | L | — | — | — | — | — | — | — | — | — | — | — | — | — |
| LIP062_1855 | — | — | — | — | — | Y | — | — | — | — | — | — | — | — | — | — | — | — | — |
| LIP062_1854 | — | — | — | — | — | — | — | — | — | — | — | — | E | — | — | — | — | — | — |
| LIP062_1853 | — | — | — | — | — | — | — | — | — | — | — | — | Q | — | — | — | — | — | — |
| LIP062_1852 | — | — | — | — | — | — | — | — | — | — | — | — | T | — | — | — | — | — | — |
| LIP062_1851 | — | — | — | — | — | — | — | — | — | — | — | — | H | — | — | — | — | — | — |
| LIP062_1850 | — | — | — | — | — | — | — | — | — | — | — | — | D | — | — | — | — | — | — |
| LIP062_1849 | — | — | — | — | — | — | — | — | — | — | — | — | V | — | — | — | — | — | — |
| LIP062_1848 | — | — | — | — | — | — | — | — | — | — | — | — | R | — | — | — | — | — | — |
| LIP062_1847 | — | — | — | — | — | — | — | — | — | — | — | — | N | — | — | — | — | — | — |
| LIP062_1846 | — | — | — | — | — | — | — | — | — | — | — | — | G | — | — | — | — | — | — |
| LIP062_1845 | — | — | — | — | — | — | — | — | — | — | — | — | A | — | — | — | — | — | — |
| LIP062_1844 | — | — | — | — | — | — | — | — | — | — | — | — | S | — | — | — | — | — | — |
| LIP062_1843 | — | — | — | — | — | — | — | — | — | M | — | — | — | — | — | — | — | — | — |
| LIP062_1842 | — | — | — | — | — | — | — | — | — | G | — | — | — | — | — | — | — | — | — |
| LIP062_1841 | — | — | — | — | — | — | — | — | — | R | — | — | — | — | — | — | — | — | — |
| LIP062_1840 | — | — | — | — | — | — | — | — | — | F | — | — | — | — | — | — | — | — | — |
| LIP062_1839 | — | — | — | — | — | — | — | — | — | E | — | — | — | — | — | — | — | — | — |
| LIP062_1838 | — | — | — | — | — | — | — | — | — | W | — | — | — | — | — | — | — | — | — |
| LIP062_1837 | — | — | — | — | — | — | — | — | — | L | — | — | — | — | — | — | — | — | — |
| LIP062_1836 | — | — | — | — | — | — | — | — | — | Y | — | — | — | — | — | — | — | — | — |
| LIP062_1835 | — | — | — | — | — | — | — | — | — | S | — | — | — | — | — | — | — | — | — |
| LIP062_1834 | — | — | — | — | — | — | — | — | — | C | — | — | — | — | — | — | — | — | — |
| LIP062_1833 | — | — | — | — | — | — | — | — | — | A | — | — | — | — | — | — | — | — | — |
| LIP062_1832 | — | — | — | — | — | — | — | — | — | V | — | — | — | — | — | — | — | — | — |
| LIP062_1831 | — | — | — | — | — | — | — | — | — | N | — | — | — | — | — | — | — | — | — |
| LIP062_1830 | — | — | — | — | — | — | M | — | — | — | — | — | — | — | — | — | — | — | — |
| LIP062_1829 | — | — | — | — | — | — | S | — | — | — | — | — | — | — | — | — | — | — | — |
| LIP062_1828 | — | — | — | — | — | — | C | — | — | — | — | — | — | — | — | — | — | — | — |
| LIP062_1827 | — | N | — | — | — | — | — | N | — | — | — | — | — | — | — | — | — | — | — |
| LIP062_1826 | — | — | — | — | — | — | — | — | I | — | — | — | — | — | — | — | — | — | — |
| LIP062_1825 | — | — | — | N | — | — | V | — | — | — | A | — | — | — | A | G | — | — | — |
| LIP062_1824 | — | — | — | T | — | — | V | — | — | — | A | — | — | — | — | G | — | — | — |
| LIP062_1823 | — | — | — | N | — | — | V | — | — | — | A | — | — | — | S | S | — | — | — |
| LIP062_1822 | — | — | — | H | — | — | T | — | — | — | A | — | — | — | S | S | — | — | — |
| LIP062_1820 | — | — | — | — | — | — | — | — | — | — | A | — | — | — | A | T | — | — | — |
| LIP062_1818 | — | — | — | Y | — | — | — | — | — | — | A | — | — | — | — | T | — | — | — |
| LIP062_1817 | — | — | — | — | — | — | — | — | — | — | A | — | — | — | G | T | — | — | — |
| LIP062_1816 | — | — | — | — | — | — | — | — | — | — | A | — | — | — | N | A | — | — | — |
| LIP062_1814 | — | — | — | T | — | — | A | — | — | — | A | — | — | — | — | T | — | — | — |
| LIP062_1812 | — | — | — | N | — | — | — | — | — | — | A | — | — | — | — | A | — | — | — |
| LIP062_1810 | — | — | — | T | — | — | — | — | — | — | A | — | — | — | N | T | — | — | — |
| LIP062_1807 | — | — | — | — | — | — | — | — | — | — | A | — | — | — | D | A | — | — | — |
| LIP062_1805 | — | — | — | H | — | — | V | — | — | — | A | — | — | — | — | A | — | — | — |
| LIP062_1804 | — | — | — | H | — | — | — | — | — | — | A | — | — | — | A | T | — | — | — |
| LIP062_1803 | — | — | — | N | — | — | V | — | — | — | A | — | — | — | S | A | — | — | — |
| LIP062_1801 | — | — | — | — | — | — | — | — | — | — | A | — | — | — | — | G | — | — | — |
| LIP062_1799 | — | — | — | — | — | — | — | — | — | — | A | — | — | — | N | T | — | — | — |
| LIP062_1798 | — | — | — | Y | — | — | V | — | — | — | A | — | — | — | N | T | — | — | — |
| LIP062_1797 | — | — | — | H | — | — | T | — | — | — | A | — | — | — | — | A | — | — | — |
| LIP062_1796 | — | — | — | H | — | — | — | — | — | — | A | — | — | — | A | S | — | — | — |
| LIP062_1795 | — | — | — | N | — | — | V | — | — | — | A | — | — | — | N | T | — | — | — |
| LIP062_1793 | — | — | — | — | — | — | — | — | — | — | A | — | — | — | — | T | — | — | — |
| LIP062_1792 | — | — | — | Y | — | — | V | — | — | — | A | — | — | — | S | T | — | — | — |
| LIP062_1790 | — | — | — | — | — | — | — | — | — | — | A | — | — | — | S | S | — | — | — |
| LIP062_1788 | — | — | — | N | — | — | L | — | — | — | A | — | — | — | S | G | — | — | — |
| LIP062_1782 | — | — | — | N | — | — | — | — | — | L | — | — | — | — | N | T | — | — | — |
| LIP062_1781 | — | — | — | H | — | — | A | — | — | L | — | — | — | — | A | T | — | — | — |
| LIP062_1780 | — | — | — | H | — | — | — | — | — | L | — | — | — | — | — | G | — | — | — |
| LIP062_1779 | — | — | — | N | — | — | V | — | — | L | — | — | — | — | D | T | — | — | — |
| LIP062_1778 | — | — | — | — | — | — | — | — | — | L | — | — | — | — | A | T | — | — | — |
| LIP062_1776 | — | — | — | H | — | — | V | — | — | L | — | — | — | — | — | A | — | — | — |
| LIP062_1775 | — | — | — | T | — | — | V | — | — | L | — | — | — | — | S | A | — | — | — |
| LIP062_1774 | — | — | — | — | — | — | — | — | — | L | — | — | — | — | D | A | — | — | — |
| LIP062_1773 | — | — | — | N | — | — | V | — | — | L | — | — | — | — | A | A | — | — | — |
| LIP062_1770 | — | — | — | — | — | — | — | — | — | L | — | — | — | — | N | T | — | — | — |
| LIP062_1768 | — | — | — | — | — | — | — | — | — | L | — | — | — | — | D | T | — | — | — |
| LIP062_1767 | — | — | — | — | — | — | — | — | — | L | — | — | — | — | S | T | — | — | — |
| LIP062_1766 | — | — | — | — | — | — | — | — | — | L | — | — | — | — | N | A | — | — | — |
| LIP062_1704 | — | — | — | H | — | — | — | — | — | — | — | — | — | — | A | A | — | — | — |
| LIP062_1703 | — | — | — | H | — | — | T | — | — | — | — | — | — | — | A | A | — | — | — |
| LIP062_1701 | — | — | — | T | — | — | V | — | — | — | — | — | — | — | G | T | — | — | — |
| LIP062_1700 | — | — | — | — | — | — | — | — | — | — | — | — | — | — | S | T | — | — | — |
| LIP062_1696 | — | — | — | — | — | — | — | — | — | — | — | — | — | — | A | T | — | — | — |
| LIP062_1695 | — | — | — | N | — | — | V | — | — | — | — | — | — | — | A | T | — | — | — |
| LIP062_1694 | — | — | — | — | — | — | — | — | — | — | — | — | — | — | — | G | — | — | — |
| LIP062_1692 | — | — | — | — | — | — | — | — | — | — | — | — | — | — | A | T | — | — | — |
| LIP062_1691 | — | — | — | N | — | — | V | — | — | — | — | — | — | — | S | S | — | — | — |
| LIP062_1686 | — | — | — | H | — | — | V | — | — | — | — | — | — | — | A | S | — | — | — |
| LIP062_1685 | — | — | — | N | — | — | V | — | — | — | — | — | — | — | N | A | — | — | — |
| LIP062_1684 | — | — | — | — | — | — | — | — | — | — | — | — | — | — | N | T | — | — | — |
| LIP062_1683 | — | — | — | — | — | — | — | — | — | — | — | — | — | — | D | A | — | — | — |
| LIP062_1681 | — | — | — | T | — | — | — | — | — | — | — | — | — | — | N | T | — | — | — |
| LIP062_1680 | — | — | — | N | — | — | A | — | — | — | — | — | — | — | — | T | — | — | — |
| LIP062_1678 | — | — | — | N | — | — | — | — | — | — | — | — | — | — | — | A | — | — | — |
| LIP062_1677 | — | — | — | — | — | — | — | — | — | — | — | — | — | — | G | T | — | — | — |
| LIP062_1676 | — | — | — | Y | — | — | — | — | — | — | — | — | — | — | G | T | — | — | — |
| LIP062_1674 | — | — | — | — | — | — | — | — | — | — | — | — | — | — | G | T | — | — | — |
| LIP062_1670 | — | — | — | N | — | — | — | — | — | — | — | — | — | — | — | T | — | — | — |
| LIP062_1669 | — | — | — | — | — | — | — | — | — | — | — | — | — | — | S | G | — | — | — |
| LIP062_1668 | — | — | — | N | — | — | — | — | — | — | — | — | — | — | — | G | — | — | — |
| LIP062_1667 | — | — | — | — | — | — | A | — | — | — | — | — | — | — | — | G | — | — | — |
| LIP062_1665 | — | — | — | — | — | — | — | — | — | — | — | — | — | — | D | T | — | — | — |
| LIP062_1664 | — | — | — | N | — | — | — | — | — | L | — | — | — | — | A | T | — | — | — |
| LIP062_0450 | — | — | — | — | — | — | F | — | — | — | — | — | — | — | — | — | — | — | — |
| LIP062_0449 | — | — | — | — | — | — | Y | — | — | — | — | — | — | — | — | — | — | — | — |
| LIP062_0391 | — | — | — | — | — | — | — | — | — | — | — | — | — | — | — | — | — | — | — |

In one embodiment the expression cassette further comprises a promoter which is operably linked to the nucleic acid molecule encoding the leader peptide.

The term “promoter” as used herein refers to a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription may also be characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors,

such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)).

A promoter may be constitutively active, repressible or inducible. If a promoter is an inducible promoter, then the rate of transcription initiation increases in response to an inducing agent or the promoter provides for gene expression in the presence of the inducing agent, but not in the absence of the inducing agent. In contrast, the rate of transcription initiation is not regulated by an inducing agent if the promoter is a constitutive promoter. Hence, a constitutive promoter is typically active under most conditions in the cell. Repressible promoters are also known.

Constitutive promoters for protein expression in yeast cells and in particular in Komagataella phaffii include, but are not limited to, the GAP (glyceraldehyde-3-phosphate dehydrogenase; Waterham et al. (1997) Gene 186: 37-44), TEF1 (translation elongation factor 1 (Ahn et al. (2007) Appl. Microbiol. Biotechnol. 74: 601-608), PGK1 (3-phosphoglycerate kinase; de Almeida et al. (2005) Yeast 22: 725-737), GCW14 (Liang et al. (2013) Biotechnol. Lett. 35: 1865-1871), G1 (high affinity glucose transporter; Prielhofer (2013) Microb. Cell Factories 12:5) and G6 promoter (Prielhofer (2013) Microb. Cell Factories 12:5).

Inducible promoters for protein expression in yeast cells and in particular in Komagataella phaffii may be promoters which are inducible by methanol. Promoters which are inducible by methanol drive gene expression when methanol is added to the culture medium. Promoters inducible by methanol include, but are not limited to, the AOX1 (alcohol oxidase 1; Tschopp et al. (1987) Nucleic Acids Res. 15: 3859-3876), DAS (dihydroxyacetone synthase; Ellis et al. (1985) Mol. Cell. Biol. 5: 1111-1121) and FLD1 (formaldehyde dehydrogenase 1; Shen et al. (1998) Gene 216: 93-102). In one embodiment, the AOX1 promoter is used.

The promoter can be specific for bacterial, mammalian or yeast expression, for example. Preferably, the promoter is functional in yeast cells. In some embodiments, the promoter is specific for expression in yeast, i.e. the promoter initiates transcription in yeast cells, but not in other cells.

In some embodiments, the promoter is a promoter that is useful in driving protein expression independently of methanol, wherein the promoter drives protein expression in a methanol-free medium. This means that the promoter is active in the absence of methanol. The expression “promoter is active in the absence of methanol” is used herein interchangeably with “promoter drives protein expression independently of methanol” and “promoter that allows an increase in protein expression in the absence of methanol”. Such promoters are disclosed in U.S. provisional application 62/682,053 and herein as SEQ ID Nos. 11-17.

The promoter may also be inducible by substances other than methanol. The isocitrate lyase ICL1 promoter is induced in the absence of glucose and/or by the addition of ethanol (Menendez et al. (2003) Yeast 20: 1097-1108). The PH089 promoter is induced by phosphate starvation (Ahn et al. (2009) Appl. Environ. Microbiol. 75: 3528-3534). The THI11 promoter is repressed by thiamin (Stadlmayr et al. (2010) J. Biotechnol. 150: 519-529). The alcohol dehydrogenase ADH1 promoter is repressed on glucose and methanol and induced by glycerol and ethanol (U.S. Pat. No. 8,222,386). The enolase ENO1 promoter is repressed on glucose, ethanol and methanol and induced on glycerol (U.S. Pat. No. 8,222,386). The glycerol kinase GUT1 promoter is repressed on methanol and induced on glucose, glycerol and ethanol (U.S. Pat. No. 8,222,386).

The promoter is operably linked to the nucleic acid molecule encoding the leader peptide, meaning that the promoter is capable of effecting the expression of the leader peptide. If the nucleic acid molecule encoding the leader peptide is operably linked to a nucleic acid sequence encoding a protein, the promoter is capable of effecting the expression of the leader peptide and the protein. In one embodiment the nucleic acid sequences operably linked to each other are immediately linked, i.e. without further elements or nucleic acid sequences between the promoter and the nucleic acid sequence encoding the leader peptide and/or between the nucleic acid sequence encoding the leader peptide and the nucleic acid sequence encoding the protein.

The expression cassette may further contain a suitable terminator sequence operably linked to the nucleic acid sequence encoding the protein. Suitable terminator sequences include, but are not limited to, the AOX1 (alcohol oxidase) terminator, the CYC1 (cytochrome c) terminator and the TEF (translation elongation factor) terminator.

The term “vector” refers to DNA sequences that are required for the transcription of cloned recombinant nucleotide sequences, i.e. of recombinant genes and the translation of their mRNA in a suitable host organism. Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together.

The term “vector” as used herein includes autonomously replicating nucleotide sequences as well as genome integrating nucleotide sequences. Vectors include, but are not limited to, plasmids, minicircles, yeast, yeast integrative plasmids, episomal plasmids, centromere plasmids, artificial chromosomes and viral genomes. Available commercial vectors are known to those of skill in the art. Commercial vectors are available from European Molecular Biology Laboratory and Atum, for example.

In a preferred embodiment the expression vector according to the invention is a plasmid suitable for integration into the genome of the host cell, in a single copy or in multiple copies per cell. The nucleic acid sequence encoding the leader peptide, optionally operably linked to a protein, may also be provided on an autonomously replicating plasmid in a single copy or in multiple copies per cell. The preferred plasmid is a eukaryotic expression vector, preferably a yeast expression vector. The expression vector may be any vector which is capable of replicating in or integrating into the genome of the host organisms. Preferably, the vector is functional in yeast cells such as Komagataella phaffii cells.

The vector can be produced by any method known in the art. For example, procedures to ligate the nucleic acid sequences encoding the leader peptide and the protein and to insert the ligated sequences into a suitable vector are known and described for example in Green and Sambrook (2012) Molecular Cloning, 4th edition, Cold Spring Harbor Laboratory Press.

The term “host cell” has its typical meaning and may include, but is not limited to, for example, a cell into which a nucleic acid molecule or vector which contains a nucleic acid sequence encoding the leader peptide of the present invention has been introduced, preferably the nucleic acid sequence encoding the leader peptide is operably linked with a nucleic acid sequence encoding a protein. Accordingly, the host cell is typically a recombinant host cell which differs from the naturally occurring cell in that it contains one or more nucleic acid sequences which are not present in the naturally occurring cell. In some embodiments, the host cell is an isolated cell. The recombinant host cell can be produced by transforming the cell with the expression cassette or the vector of the present invention according to methods known in the art. Methods for transforming and culturing Komagataella phaffii cells are for example described in Pichia Protocols, 2nd edition 2007, edited by James M. Cregg, ISBN: 978-1-S8829-429-6.

In one embodiment the host cell is a yeast cell. Suitable yeast cells may be selected from the genus group consisting of Pichia, Candida, Torulopsis, Arxula, Hansenula, Ogatea, Yarrowia, Kluyveromyces, Saccharomyces, Ashbya and Komagataella.

In one embodiment the host cell is a methylotropic yeast cell. The term “methylotrophic yeast,” as used herein includes, but is not limited to, for example, yeast species that can use reduced one-carbon compounds such as methanol or methane, and multi-carbon compounds that contain no carbon bonds, such as dimethyl ether and dimethylamine. For example, these species can use methanol as the sole carbon and energy source for cell growth. Without being limiting, methylotrophic yeast species may include the genus Methanoscacina, Methylococcus capsulatus, Hansenula polymorpha, Candida Komagataella phaffii and Komagataella phaffii, for example. Preferably, the host cell is a Komagataella phaffii cell. In one embodiment the Komagataella phaffii strain is the auxotrophic strain GS115 which has a mutation in the his4 gene and is therefore unable to synthesize histidine.

In the method for producing a protein the host cell comprising the expression cassette or the vector of the present invention is cultured under suitable conditions, before the protein is obtained. The suitable conditions are those that permit expression and secretion of the protein. Suitable conditions are well-known to the person skilled in the art and include cultivation in the batch mode, the fed-batch mode and the continuous mode.

The host cell may be cultured on an industrial scale which may employ culture medium volume in a of at least 10 litres, preferably of at least 50 litres and most preferably of at least 100 litres.

The host cell may be cultured under growth conditions to obtain a cell density of at least 1 g/L cell dry weight, more preferably at least 10 g/L cell dry weight, preferably at least 20 g/L cell dry weight.

The protein produced by the host cell may be obtained by any known process for isolating and purifying proteins. Such processes include, but are not limited to, salting out and solvent precipitation, ultrafiltration, gel electrophoresis, ion-exchange chromatography, affinity chromatography, reverse phase high performance liquid chromatography, hydrophobic interaction chromatography, mixed mode chromatography, hydroxyapatite chromatography and isoelectric focusing.

The leader peptide of the present invention effects the secretion of a protein which is operably linked to the leader peptide. The term “secretion” as used herein refers to the translocation of a protein across both the plasma membrane and the cell wall. Preferably, the protein is present in the supernatant of the host cells due to the secretion.

Preferably, the use of the leader peptide of the present invention increases the secretion of a protein from the host cell. The protein is operably linked to the leader peptide of the present invention. The secretion is increased in comparison to the secretion of a protein which is operably linked to the leader peptide of mating factor alfa (MFa) from S. cerevisiae. The secretion is increased in comparison to the secretion of a protein which is operably linked to the leader peptide of mating factor alfa (MFa) from S. cerevisiae by at least 2%, preferably at least 5%, more preferably at least 8% and most preferably at least 10%. The secretion is increased in comparison to the secretion of a protein which is operably linked to the leader peptide of mating factor alfa (MFa) from S. cerevisiae by 2% to 15% or by 5% to 12% or by 8% to 10%. An increase

An increase in the secretion of a protein can be determined by determining the amount of said protein in the supernatant of a host cell of the present invention and in the supernatant of a control cell, e.g. a cell in which said protein is operably linked to the leader peptide of mating factor alfa (MFa) from S. cerevisiae and comparing these amounts.

The following examples are provided for illustrative purposes. It is thus understood that the examples are not to be construed as limiting. The skilled person will clearly be able to envisage further modifications of the principles laid out herein.

### Examples

1. General Method for Komagataella phaffii (Pichia) Expression

Leader sequences were cloned upstream of the gene of interest (for example lipase, amylase, or xylanase) in the pPlCz backbone (Thermo Fischer). The expression of the gene of interest is regulated by the methanol-inducible AOX1 promoter which is present in the pPlCz backbone or by the methanol-free constitutive promoter according to SEQ ID No. 11 cloned into the pPlCz backbone to replace the AOX1 promoter. Expression vectors were transformed into the Komagataella phaffii strain X-33 and screened for transformation by zeomycin selection as described in the User Manual for pPICZ A, B and C, Rev. Date: 7 Jul. 2010, Manual part no. 25-0148. Individual colonies of the strain transformed with the plasmid comprising the methanol-free constitutive promoter according to SEQ ID No. 11 were grown in microtiter plates in YPD medium (1% yeast extract, 2% peptone, 2% dextrose in sterile water). Individual colonies of the strain transformed with the plasmid comprising the AOX1 promoter were grown in microtiter plates in BMMY media (2% Peptone, 1% Yeast Extract, 1.34% Potassium Phosphate, pH 6.0, 100 mM Yeast Nitrogen Base (without Amino Acids), 0.4 μg/mL Biotin, 0.5% methanol). Supernatants were assayed at 24 or 48 hr for the presence of secreted enzyme by activity and protein gel analysis.

2. Expression of Lipase A

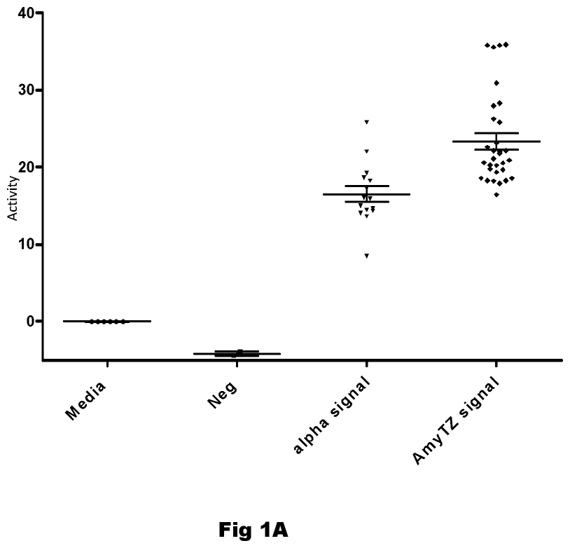

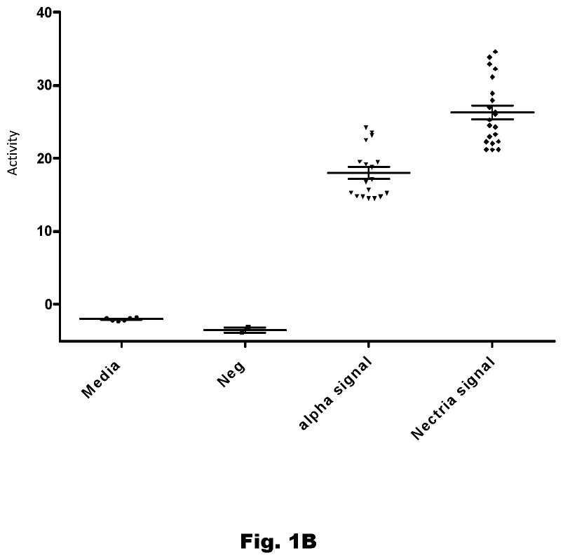

Three leader sequences (alpha factor, AmyTZ, Nectria) were tested for their ability to aid secretion of lipase A in K. phaffii Lipase expression was driven by the methanol-inducible AOX1 promoter. Individual transformants were grown in microtiter plates and expression was induced using methanol for 48 hr. Supernatants were tested for relative lipase activity by incubating the supernatants with p-octanoate as substrate at a temperature of 30° C. and a pH of 7.5 for 10 minutes. FIGS. 1 a) and b) show that the fusion of either the AmyTZ leader sequence according to SEQ ID No. 2 (a) or the Nectria leader sequence according to SEQ ID No. 3 (b) with lipase led to more transformants with higher levels of active secreted lipase than the alpha factor leader sequence. Media only or a K. phaffii strain with the empty pPlCz vector only (Neg) were used as controls.

3. Expression of Xylanase

Three leader sequences (alpha factor, AmyTZ, and the native xylanase leader sequence) were tested for their ability to aid secretion of the xylanase according to SEQ ID No. 21 in K. phaffii. Xylanase expression was driven by the constitutive promoter according to SEQ ID No. 11. Individual transformants were grown for 24 hr in microtiter plates. Supernatants of four individual transformants were tested for the presence of xylanase by protein stain gel analysis.

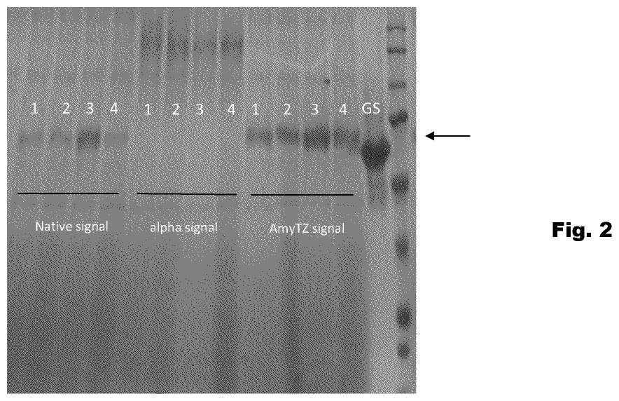

FIG. 2 shows that the fusion of the AmyTZ leader sequence led to higher levels of secreted xylanase than either the alpha factor or native leader sequences. A K. phaffii strain with the empty pPlCz vector only (Neg) and a purified xylanase (gold standard; GS) were used as controls.

4. Expression of Amylase

Two leader sequences (AmyTZ and alpha factor) were tested for their ability to aid secretion of the amylase according to SEQ ID No. 19 in K. phaffii. Amylase expression was driven by the constitutive promoter according to SEQ ID No. 11. Individual transformants were grown for 48 hr in microtiter plates. Supernatants of six individual transformants were tested for the presence of amylase by protein stain gel analysis.

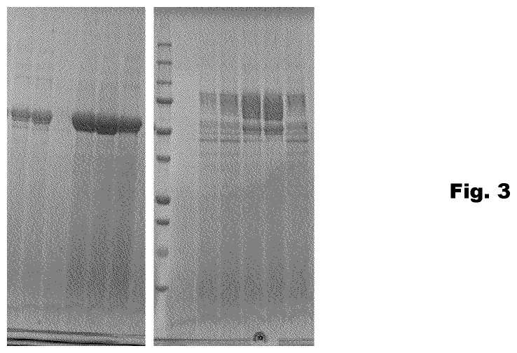

FIG. 3 shows that the AmyTZ leader sequence (left) led to higher levels of secreted amylase than the alpha factor leader sequence (right). 5. Expression of lipase B

Two leader sequences (alpha factor and AmyTZ) were tested for their ability to aid secretion of lipase B in K. phaffii. Lipase expression was driven by the methanol-inducible AOX1 promoter. Individual transformants were grown in microtiter plates and expression was induced using methanol for 48 hr. Supernatants were tested for the presence of lipase by protein stain gel or by relative lipase activity using p-octanoate as substrate at a temperature of 30° C. and a pH of 7.5 for 10 minutes.

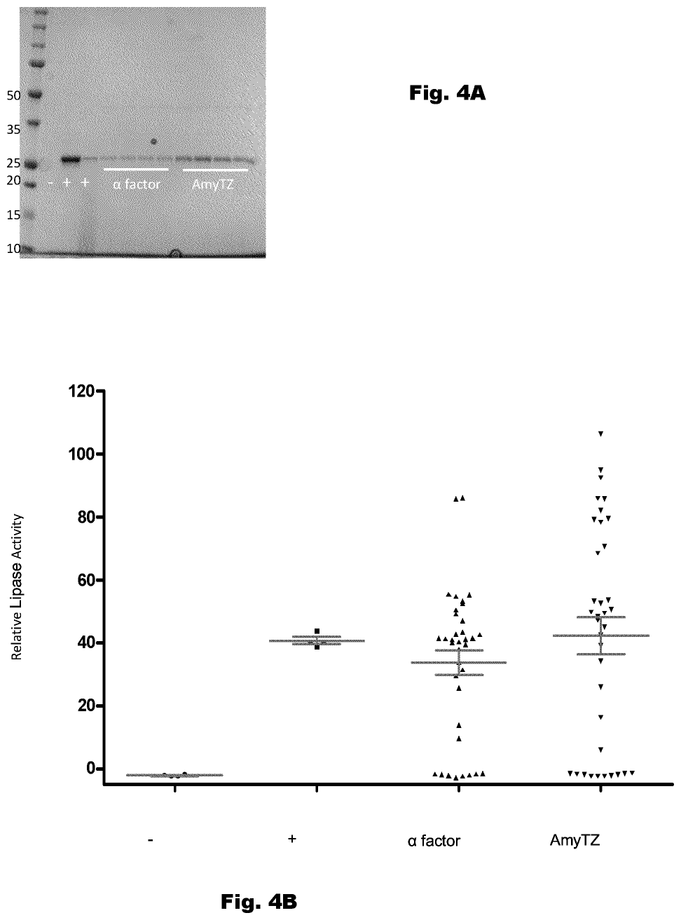

FIG. 4 shows that the fusion of either the AmyTZ signal led to more transformants with higher levels of active secreted lipase than the alpha factor leader sequence. A K. phaffii strain with the empty pPlCz vector only (Neg) or a K. phaffii strain known to express lipase C (pos) were used as controls.

1. An isolated leader peptide selected from the group consisting of:
(a) a peptide comprising the amino acid sequence according to SEQ ID No. 1 or a functional variant thereof;
(b) a peptide comprising an amino acid sequence selected from the group of SEQ ID No. 2, SEQ ID No. 3, SEQ ID No. 4 and SEQ ID No. 5, or a functional variant thereof; and
(c) a peptide comprising an amino acid sequence which is at least 80% identical to the amino acid sequence according to any one of SEQ ID Nos. 1, SEQ ID No. 2, SEQ ID No. 3, SEQ ID No. 4 and SEQ ID No. 5.

2. An isolated nucleic acid molecule comprising a nucleic acid sequence which encodes a leader peptide according to claim 1.

3. The isolated nucleic acid molecule according to claim 2, wherein the nucleic acid sequence is selected from the group consisting of:
(a) a nucleic acid sequence encoding a peptide comprising an amino acid sequence according to any one of SEQ ID Nos. 1, SEQ ID No. 2, SEQ ID No. 3, SEQ ID No. 4 and SEQ ID No. 5;
(b) a nucleic acid sequence comprising the sequence according to any one of SEQ ID Nos. 6, SEQ ID No. 7, SEQ ID No. 8, SEQ ID No. 9 and SEQ ID No. 10;
(c) a nucleic acid sequence which is at least 80% identical to the nucleic acid sequence according to any one of SEQ ID Nos. 6, SEQ ID No. 7, SEQ ID No. 8, SEQ ID No. 9 and SEQ ID No. 10; and
(d) a nucleic acid sequence hybridizing under stringent conditions with a complementary sequence of the nucleic acid sequence according to any one of SEQ ID Nos. 6, SEQ ID No. 7, SEQ ID No. 8, SEQ ID No. 9 and SEQ ID No. 10.

4. An expression cassette comprising the nucleic acid molecule according to claim 2 operably linked to a nucleic acid sequence encoding a protein.

5. The expression cassette according to claim 4, wherein the protein is an enzyme, a peptide, an antibody or antigen-binding fragment thereof, a protein antibiotic, a fusion protein, a vaccine or a vaccine-like protein or particle, a growth factor, a hormone or a cytokine.

6. The expression cassette according to claim 5, wherein the enzyme is selected from the group consisting of lipase, amylase, glucoamylase, protease, xylanase, glucanase, cellulase, mannanase and phytase.

7. The expression cassette according to claim 4, further comprising a promoter operably linked to the nucleic acid molecule.

8. A vector comprising the expression cassette according to claim 4.

9. A host cell comprising the expression cassette according to claim 4.

10. The host cell according to claim 9, being a yeast cell.

11. The host cell according to claim 10, wherein the yeast cell is selected from the group consisting of Komagataella, Candida, Torulopsis, Arxula, Hansenula, Ogatea, Yarrowia, Kluyveromyces, Ashbya and Saccharomyces.

12. A method for producing a protein in a host cell, comprising the steps of:
(a) providing the host cell according to claim 9;
(b) culturing the host cell under suitable conditions; and
(c) obtaining the protein.

13. A method of secretion of a protein from a host cell and/or for increasing the secretion of a protein from a host cell comprising using the leader peptide according to claim 1 for the secretion of a protein from a host cell and/or for increasing the secretion of a protein from a host cell.

14. An expression cassette comprising the nucleic acid molecule according to claim 3 operably linked to a nucleic acid sequence encoding a protein.

15. The expression cassette according to claim 5, further comprising a promoter operably linked to the nucleic acid molecule.

16. The expression cassette according to claim 6, further comprising a promoter operably linked to the nucleic acid molecule.

17. A vector comprising the expression cassette according to claim 5.

18. A vector comprising the expression cassette according to claim 6.

19. A vector comprising the expression cassette according to claim 7.

20. A host cell comprising the expression cassette according to claim 5.

21. A host cell comprising the expression cassette according to claim 6.

22. A host cell comprising the expression cassette according to claim 7.

23. A host cell comprising the vector according to claim 8.

24. A method for producing a protein in a host cell, comprising the steps of:
(a) providing the host cell according to claim 10;
(b) culturing the host cell under suitable conditions; and
(c) obtaining the protein.

25. A method for producing a protein in a host cell, comprising the steps of:
(a) providing the host cell according to claim 11;
(b) culturing the host cell under suitable conditions; and
(c) obtaining the protein.

26. A method of secretion of a protein from a host cell and/or for increasing the secretion of a protein from a host cell comprising using the nucleic acid sequence according to claim 2 for the secretion of a protein from a host cell and/or for increasing the secretion of a protein from a host cell.

27. A method of secretion of a protein from a host cell and/or for increasing the secretion of a protein from a host cell comprising using the nucleic acid sequence according to claim 3 for the secretion of a protein from a host cell and/or for increasing the secretion of a protein from a host cell.


SEQUENCE LISTING (23 sequences)

SEQ ID NO: 1 - protein, 31 aa, artificial
  leader peptide
  misc_feature (13)..(13): Xaa can be any naturally occurring amino acid
  misc_feature (30)..(30): Xaa can be any naturally occurring amino acid
MRLLPLLSVVTLXAASPIASVQEYTDALEXR

SEQ ID NO: 2 - protein, 31 aa, Fusarium solani
MRLLPLLSVVTLTAASPIASVQEYTDALEKR

SEQ ID NO: 3 - protein, 31 aa, Fusarium solani
MRLLPLLSVVTLAAASPIASVQEYTDALETR

SEQ ID NO: 4 - protein, 31 aa, artificial
  variant of leader peptide
MRLLPLLSVVTLAAASPIASVQEYTDALEKR

SEQ ID NO: 5 - protein, 31 aa, artificial
  variant of leader peptide
MRLLPLLSVVTLTAASPIASVQEYTDALETR

SEQ ID NO: 6 - DNA, 93 bp, artificial
  nucleic acid sequence encoding variant of leader peptide
  misc_feature (37)..(39): n is a, c, g, or t
  misc_feature (88)..(90): n is a, c, g, or t
atgaggctgcttccactgttgtccgtcgttacattgnnngccgcttccccaatcgcctctgtccaggaatacaccgacgctttggaannnaga

SEQ ID NO: 7 - DNA, 93 bp, Fusarium solani
atgaggctgcttccactgttgtccgtcgttacattgactgccgcttccccaatcgcctctgtccaggaatacaccgacgctttggaaaaaaga

SEQ ID NO: 8 - DNA, 93 bp, Fusarium solani
atgaggctgcttccactgttgtccgtcgttacattggctgccgcttccccaatcgcctctgtccaggaatacaccgacgctttggaaacaaga

SEQ ID NO: 9 - DNA, 93 bp, artificial
  nucleic acid encoding leader peptide variant
atgaggctgcttccactgttgtccgtcgttacattggctgccgcttccccaatcgcctctgtccaggaatacaccgacgctttggaaaaaaga

SEQ ID NO: 10 - DNA, 93 bp, artificial
  nucleic acid sequence encoding leader peptide variant
atgaggctgcttccactgttgtccgtcgttacattgactgccgcttccccaatcgcctctgtccaggaatacaccgacgctttggaaacaaga

SEQ ID NO: 11 - DNA, 1501 bp, artificial
  promoter pSD001
tccagtgtagcactaaaatctaatatcttcggctttatacttttttgttcatccgaaagcttacgaacaattctttctcctgttttattgtggatatagacaatttcgtcagtttcttggagagaagagttatttccggttttggctggccctataaacgggttcttggatttggatctagtaataaaaatgtcactgtcattctcggagctgaactttgtgttgtacgaagatgggttgttccactgttttgccagctcttcattgatgattttcttagtgggtgttcttggaggttcacgttgcctataatcttgacgttcttcttcatcactatcgatgccatcaaaattaagcgtccttattgcaggcttttgtgatttcaactgcaatccttctatctcttcatcagagctttcgaactgaatactatcactcaaaactggcgacattgcacatttccgcaaaccatttcgggaatctatgctagctcttctagacgataaagaacgaccggaaccaatacggggttgtgcaggtgggaataaatatgttggtttggattcttgacgtgaagaaggtattctagtcgatgaagtggttgataaggatatggcgtcactgagttgttttcttttcctatgttgcggtgttgggtcaggagttaattgattcacctccataactctggaatttcttgaatgtggggttttcagatgggcatctttcttgacggggttgtgagtaacggaggaacctggtgtcttgggtgtgaacggtgtttgagcctgtacgcggttacttctgggcggagtactcggagtcatgagagccattgattagaaggtgaatgagggagtcaccactctaagcaaacaaaatgaggtcgaagcaaaaaataaagtaaagtagcacttctggcaggttagatcaaagagtgacgggagatttgaagatggctggtttttccttagtcttggaagaggtttgtgtgggtatcagcgaatattccccgattaggcaaattagttgcattgaaattaacacgacatggtgatttgtggtaacaaatatctattggtggttggtgtgtgggtgtaatagtggtcgtgtcatgatgatggtgttcaggtgttgtcatagatcggtcttcagtaagagaaggaagcttggtgacgatcacagctatgatgtaatagaaattgctaagcaattgtgaggtgtgatgtattttgcagagcaattgtgcggtacaacggggtgttattgtcttcacaaggcatttattgcgaatttcgtagttgaaagaatattttagcacagggtgcttgacccctattgttgctcgctaaaccatgattgctaaatgatgacatagcaatcactttactaagattgctataaggacacctttcttagtataaatggacactcttttcccctgctaaacttcttttatttttcacacttaaacagttacaaaacacaaacacaactagaa

SEQ ID NO: 12 - DNA, 1501 bp, artificial
  promoter pSD002
gtgctaaaatctgaggtttacaagctgtgatgttcccctaagatctcacaatcgaacaatcgcgaagccaatgcaagttgtttaaggggaaacgactcactattcctgaaattagtattcaaaacttggtccggaagaacaatgaggcggccgttaaaatactcacgtaaacggtgtctacaagcgcattaaaatccgtttgaattcaagcaaaagccaccagaggcttatgcttggttatacccagcattgacctttggtatgagcatctgaaaaacaaccaggtgttgcaaagttaaacatccttctttgttcatatagaacccactattcatggtactccccaatcgaatttcacattctggttttgaaattacacaccacgttagcttataagatttcatataacttattgatatacggtttccattgttcgaatagttgaggttgtatgtaattcgattgaaggggccatttttgtttcctacttttcctgggagcttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaa

SEQ ID NO: 13 - DNA, 1500 bp, artificial
  promoter pSD003
tcacattcatagcatctctcgcctgcaatagcttccacgataggaatatctgtgaaagtgaacatgctatttcgatgatataagactttaagatctggcatgtttgtgttggaggttaccctggggtcaataaccctaattatctccttcactaaaaatgatgaagattcttcggattcgtttttgaacagagttaatgccatttcttcgtcaatagaaaaatcaatatctggtatctcatcttttacatattgaggatttagttttcttccctttggatagtacattatgatcaatgtattcctgtctttattgataaagtattggcattctgcttcttgtacacctttgaattgtttgtctggaagtgactgacatttttccacattgctaacggtttggcacgaattacatctaaataaaatgtcttctccggattcgtgtattaagtgatactccaatgataaatccccacctatcgaaccagaatcggcattggccacagtcacaggtaactttaggtcttgaaaaatccttctataggcttcattgacattgtcataagacttaagaccatcttctttggtcaagtcaaaagaataggcatctttcatgagaaactctcgtcctctcaacaaacctcccctaggtctcaactcatctctatatttgcgggaaatttggtacacgagaaggggtaaatctttatatgacgaacataagtcaccaactaagtttgtgatttcctcttcacaagttggcactaaacagtagtctctatccttggagtctttgaacttgaacaattcattgttgtcccatctcttagttctctcccataaatgcttggaagacaggctacttaattccatttccagcccaccagcctgatccattcttttcctaattacattttgaagctttttataggtacggagtcctaatggaagccagtgaactattcctgctgcaggctggtaaataaaccttgattgaaggagcatatcatgagtagtaaggtcctttacagaaaatagtttacttccttgaagagaagtagaataaaacctcatgttgggtctccatgaaaggttcaaaggcattgatcctttaggtacttcaggatgtttaagtcatcaaactgtccatcaaaggtagtatagtatttaccatctagatagtgatgtatgggtgtaacacaacatttaaatgttgtaaattaacattaggactgagtccggagatgctattgtcacctaaatctattagaaagcacttcagttatatcatcgatagaggtttgaagataaacctattgttgataaataaccccattacccgtttacgtagcaaggttcaaaaatttgcttagatcggagctaaaaattcgactgacttctttcgaaaatgtggattatgcaagcaacgttgctatcggaatagtatataaggtcgatctgccccattacaaattgtaaagcaacaaacatcctacgcaaa

SEQ ID NO: 14 - DNA, 1500 bp, artificial
  promoter pSD004
tcagtttcacggttatgtgagctgtctccgcgtgaggcagtaacctctgtgtcatggatacaggctggtacacatttggcagtaggaacacaatctggtttagttgaaatatgggacgccacgacgtccaaatgtacaagatcaatgactgggcattcggcccgaacctcagcgctgagttggaaccgtcatgttttgagttctggttcaagagatcgcagtatcttacatcgggatgtacgtgcagcagctcactatacaagtcgcattgttgaacaccgccaagaggtttgtggcttacgttggaacgtggatgaaaacaagctggccagtggttccaatgataaccgtatgatggtatgggatgcactgcgtgtagaacagccccttatgaaagttgaagagcatactgcggctgttaaggcgttggcatggtcacctcatcaacgtggaatactggcttcgggtggaggtactgctgacagacgtatcaaggtgtggaatactttaacaggatccaagctgcacgatgttgatactggatctcaagtttgtaatctcttgtggtctcgcaattctaatgaattggtaagtactcatggatattctcgaaaccaagtcgttatttggaaatatccgcaaatgaagcaactagcatctttgactggtcatacttatcgagtcctttacctttccatgtcacctgatggaactacagtcgtaacgggggctggagacgaaactttaagattttggaactgtttcgagaagtcacgacaaagcggaggaggatcaatattactagacgcttttagtcagcttcgttaaattaccaccaaatttggtgcaaaagggcccatatggtgctacaaccaaaggaactttctaattttgataatgatgtcatttctctcatcgggatgaaaatagaagtcgaaaggatttttgtcactatttcaagccccacctgcagctggcagcatttctattgtttatgcattgtcatttatgggaaaactaagaaagttcctctccacccggactccactggtaaatatgcgatatcggaatcatgaccaacccatattttgatcctaatcatttcggttctagtctccgatcggactccgtaaaactgcggagtgaactccaacggagaatactgcagccaatctcatatttcatttgttatttgtccctcaactgtctcgataaggtcatctgtgtttgactagatgttcgtcattggcatgtcaaacaaggctagaccttacaatcatctcttacgaatgtaagtgaatgtaactatattttccttgctactttaacgaggttaaccaacccccgcacatccccacaccaccgctcttgataagcatctccgaaaatgcatgacgcgacaacttcaagcatgttgtatttactgagttttcagcctcactatcgatacctctataaatagaggcactttcgtctcttctccctccccacaagaaacca

SEQ ID NO: 15 - DNA, 1500 bp, artificial
  promoter pSD005
agaagtactgttatgaatcgatcgacgtgacatgttgttgatggttctgacttcttgatgtccgcgttttctgtctctcaatagtggtgttcgggggaagtatggttctaatacttaacaggtaagatggttgcaatgagcacctggtaaagcaacttgaatttcctgccctgtctccgttaagttatattcgactcaaggtccttgcttcctgtctgttctgtaaaacttccctttggtgtcttctatatcaactttaaaaacaaggtagtgtgtcgagcgatagtactgtgtctttttccctatgaaaaaaatcgcaccatccaagacttctcaccttcaacagcttcaacatcatgttcggtccttttagagctacgctggtcgatctaggaggtctgctatggaaacgtccttggagaatgtccaaaccacagaaatatagactccgcaaaagaatgcaacttgtagactccaatatcgacattatttaccagggactgactgaggagggtctgtcttgcaaagtgatagataacttgaaacaaaacttcccaaaggagcatgaagtgctccccaaaaacaagtataccgtgtttaacaagacagccaaaaactatagaaagggtgttcatttggttccaaaatggaccaagaagtctttgagagagaaccccgagttcttctaattgcacatttcttcctgttcatagattatcccacacatagttgctcacaaaaaaatcactataattttcctccaccggcagtatatcactaacacctttatctttattgtagattataatctgatctttatccttagatgtatctatcatcaaccccatgctcttgaaaagcttgagtcttaacactgtcgaatcgtagttttcttgtagatcattcgatatcactgctttttcttgctcttctaattcgttgagattctgggtcaaactagagattgaattctgaaggtgattcatgttcatctccagatctgttattgattttgctaatttaaatttttcgtgttcaagctcttcgatactctttagggtctgttgacggtcttctgtttccaataattgcttgttgaactctttaagttcgtctctctgtttactgatacgtgacaacaaatctagctggtgatcgagtttaagtttccgtttggagctcaacagagaaagattttcattaatttggttgatagtttgcacgtccggttcgatctgaaaattctctatagtcgacctgattaaggacacagtctcttgaagatcggacattggatttatggagaagggagatcaaagcggaaccagttgcactgtttacctttccagtcgagatacttatcccacagggccctcactttccaggcagaagtcacctaggaggcgcatccctccgtttgcttccctcgcgacaaactcccctgtaaaagaaaacttcactgaatcgtacacctaatcatacgacactaacacagatata

SEQ ID NO: 16 - DNA, 1500 bp, artificial
  promoter pSD007
gtcctttccaaatttttggttgaaggcatcgcttaaattatgagcaggatcggtggaaataagcaggtatttcttgttaggattgtgaagggcaagctggatagatatagaagaagatgtcgtggttttaccgacaccccccttacctccaacaaagatccacttcagcgattcgtggttcacaattgatcgcaaacttggctctgcctcaatatccatggttgatgtctagttgagtggcgtttgtggtctcttgatgagttcaaggcgaaagaatatgataggaaagcatggtttgaacttttcgcgaaagaaggaatactgttccgcgagaaactccccggtgccagaaccttccattgaggttaatcggtgggaggtgttcgaatgacaatgtcagacaaggcgaacacgtcttgtgacaccagctggactaagaagattcggtatgcaccgaagaagaaggccgtgtctcaattggcaactttgcaacaaactacggaggaaaagtctcacaagcttttaaccaagttgaatcacgacgacaacgataaagaaatcctcaaccatctaacacatgaagtacaaagtagaaatgtgatcttattggacaaactagaggagctcaacaaggaactgggctggattaaagaccgaaaatgaggaaccatgagcactgggcgtttccagaaaaactgcaaccaacgatgggaaaatgataccacactactatggtcaccccacattgtgaaatttcaaaccaaaaaagatcaaccccataattccccagagggttttcccaacaattttccaacggacttgataatgagtcagatcatttgagcatattcatcttaccccttattccgtgacaatttacctattccattcaaagcatacggtatcccgtgaccttctcatggagatcattctccaccgatacagcatatacacagatatacccaactaatatcaattggaccttgatatggtcgaccttgatggtcccgtccaaccttaaaacttagtttaatgctatactttcgccttgaaccaaatctgtctccccctcaatcatctctatgcaagaaggtcaacactgattacgtgagcaacagccagcaatcgttcgagtccccgccaaaaaaggcggagttactgctccttgtgaccacaccccctgagaccacgtccctaaacgatccttgtcggttccttcgtccaattggcaattgccacgcatacgtgaatcgttattgtttcgcctaccttgcgtcattcgttccagaatgttcgacatactcctctagaacataccgtcacaccaccatcttaagttatcttcacgtgaccatgacgtacattgtagttgactaccccattctcatcattccgatgcggccaaaaatctctatataaagaccgtatcccctaatattctcttcttgttaagacattaacttagttaattcaccaattactcacttataaacaaacaaa

SEQ ID NO: 17 - DNA, 1500 bp, artificial
  promoter pSD008
gtttctcttggggagatacttttttcgcgtgctcctccgtgcggaacttccttctgagcttctacctctcagattagtctaatcgcatcaggaataagactgagaatgcttttaaggagaggcttgagattggctaattgcgttccgaagtactctttcaaaaggagttatacccctctcaactacgattctctaaagaattatcgtaggcatgctcaggcgcctcaaccccatcagtttgacgccactagatgggaccaacaaccagttactaatgagcaaggagtaatactcccatccgactcaattgcaaacattctgagacaaccaactctggtcatagaacggcaaatggaaatgatgaatatatttttaggatttgagcaggcgaaccgatatgttatcatggatcctacaggaagtattttgggttacatgctagaaagggatctgggcatcaccaaagctatattgagacagatctaccgtttgcatcgaccttttacagtggatgtaatggatactgcaggaaatgtattaatgacaatcaagaggccgtttagtttcatcaattcgcacatcaaagctatattaccccctttcaggaacagcgacccagacgaacatgtaattggagaatccgttcaaagctggcatccttggagacgaagatacaatctatttacagcacaaattggcgaaaaggacactgtctacgatcagttcgggtacattgacgcaccgtttctttcctttgagtttcctgtactttcagaatctaggcaaacgctaggtgctgtctctagaaacttcgtgggctttgcaagagagcttttcacagatacaggagtttacatcatccgtatggggcctgaatcttttgtagggctagaagggaactacgggaacaatgtggcccaacatgcccttacgctggaccaaagggctgtattattagccaatgccgtttcaattgactttgattacttttctaggcactcgtcacacagtggtggcttcattgggtttgaggaatagacagggtctcgtcaactcagctcctgccaccaaaccaatcattgatcaacgagcacacttttgtccacgtgagatcgctttcgcttgcagaaagagcaatgcatgaaaacggcaaacgcaaaacgagcaaaaaaacgagtaaataactacaatttcaccaccaacagggtcaaagagcttttgagacactataaaaggggccctttccccccaggttccttgaaatcctcattcaattatgttttttactcataatttgactcaattggcatcttcttctttgttcatatacagtaattgatatgacgcttagtcattattagtgttctcgactagcagtggcgaaaaaagggggagttattttctagaaccgaccgcaaactataaaagaaagctgcccctcatatacctttcgaattctttattttctgtgtttcttccctatttaacatctacacaaaa

SEQ ID NO: 18 - DNA, 1302 bp, artificial
  amylase
ggtgtcatggttcacttgttccaatggaaatacaatgacattgccaacgagtgtgagaaggttcttggtccaaaaggttatgaagctgtgcagattactccacctgctgagcacttgcaaggatcttcctggtgggttgtctaccaacctgtttcctacaagaacttcacttctctgggaggtaacgaggctgaattaaaatctatgatcgctagatgtaaggctgccggtgtcaagatttacgctgacgctgtattcaaccaattggctggtggatcaggtgtcggtacaggtggatctagctacaacgccggttccttctcatacccacaatttggctacaacgatttccatcacgctggtccattgaccaactatactgacagaaacaatgtgcaaaacggtgccttgcacggtttgccagacttggataccggatctgcctatgttcaagaccagcttgctacctacatgaagaccttgagtggctggggagttgctggttttcgtcttgacgcagctaagcacatgtctgttgccgatttatcggccattgtctcaaaggctggtaacccttttgtctactccgaggttattggtgccactggtgagccaatccaaccaggtgaatacacaggaattggtgcagttactgaattcaaatacggtactgacctagcttccaacttcaagggacagattaagaacttaaagtctatgggcgagtcatggggtttgcttgctagtaacaaggctgaagtctttgttgtcaaccacgaccgtgagagaggtcatggaggtggaggtatgttgacttacaaggatggtgctttgtacaatctggccaacatcttcatgctggcttggccatatggtgcttatcctcaggttatgtccggttacgacttcggtaccaacactgatattggtggtccatctgctaccccttgttcttccggttcttcctggaactgcgaacacagatggtctaacatcgctaacatggtctctttccacaatgctgcccaaggaacttccatgaccaactggtgggacaatggtaataaccagattgctttcggtagaggtgccaaagcttttgttgtcatcaacaatgagtcttccactttgagaaagaagttgcaaactggtctgccagctggtgagtactgtaacattttggccggtgatgctttgtgttctggttccaccatcaaggttgatgcttctggtatggctaccttcaacgttgcaggtatgaaggctgcagctatccatatcaatgccaagccagattcctaa

SEQ ID NO: 19 - protein, 433 aa, artificial
  amylase
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGPLTNYTDRNNVQNGALHGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLRKKLQTGLPAGEYCNILAGDALCSGSTIKVDASGMATFNVAGMKAAAIHINAKPDS

SEQ ID NO: 20 - DNA, 1161 bp, Penicillium sp.
gccggcttgaacaccgccgctaaggctattggtttgaagtacttcggtactgctactgacaacccagagttatctgatactgcttatgaaacccagctaaacaatactcaagatttcggccagttgactccagcaaactctatgaagtgggacgccactgagcccgagcaaaatgtcttcactttctctgctggtgaccaaatcgctaatttggcaaaagctaacggtcagatgcttagatgtcacaatttggtttggtacaaccagctgccatcctgggtcacctcgggatcatggaccaatgagacactcctggcagccatgaagaaccacattaccaacgtcgttacccattacaagggtcagtgctatgcttgggatgtggtcaacgaagccttaaacgacgatggtacttaccgttccaacgtcttctaccaatacattggtgaagcttatatccctatcgctttcgccactgctgccgccgccgaccctaacgccaaactttactataacgattacaacatcgaataccctggtgctaaggctactgctgcccaaaacctggttaagttggtgcaatcctacggagctagaattgatggtgtcggtttgcagtcacactttattgttggtgagactccttctacttcttcccaacagcagaatatggctgcctttacagcattgggcgttgaggttgctatcaccgaattggatattagaatgcaattgccagagaccgaggccttgctgactcaacaggccactgactaccaatcaactgttcaagcttgtgccaacaccaaaggttgtgtcggaattaccgtttgggactggaccgataagtatagttgggttccatctactttttccggttacggggacgcttgcccttgggacgctaactatcagaagaaaccagcttacgagggtatcttgaccggtctaggtcaaacggtgacctccactacatacatcattagcccaaccacttctgttggtaccggtaccacaacttcttccggaggttccggtggaaccactggagttgctcaacattgggagcagtgtggtggattgggttggaccggtccaactgtttgtgcctctggttacacttgtactgtcattaatgaatactattctcaatgtttgtaa

SEQ ID NO: 21 - protein, 386 aa, Penicillium sp.
AGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 22 - DNA, 954 bp, Fusarium solani
gccattactgcttctcaattggactacgaaaacttcaagttttacatccagcacggtgccgctgcttactgtaactccgaaactgcctctggtcaaaagatcacttgttccgacaacggttgcaaaggtgtcgaagctaacaacgctattattgtcgcctctttcgttggaaaaggtactggtattggtggttacgtttctactgataacgttagaaaggagatcgttttgtctattagaggttcttccaacattcgtaactggttgactaacgtcgacttcggacaatcctcttgttcttacgttagagattgtggagttcacactggtttcagaaatgcttgggacgagattgcccaaagagctagagacgctgtcgctaaagctagaactatgaacccatcttacaaggttatcgctactggtcactctttgggtggtgctgttgccactttgggtgctgctgatttgagatccaagggtactgccgtcgatatctttacttttggtgccccaagagttggtaacgctgagttgtccgctttcatcactgctcaggctggtggtgagttcagagttactcacggacgtgatccagttccacgtttgccacctatcgtcttcggttacagacacacctctccagagtactggttggctggtggtgcttccaccaagactgattatactgttaacgatatcaaggtttgtgaaggtgccgctaacttggcctgtaatggtggtactttgggattggatatcattgctcatttgagatacttccaagacactgacgcctgtactgctggtggtatctcctggaagagaggtgacaaagctaagagagatgagattccaaaaagacaagaaggaatgactgatgaggagttggaacaaaaactgaacgactatgtcgccatggataaggagtacgttgagtccaacaagatgtaa

SEQ ID NO: 23 - protein, 317 aa, Fusarium solani
AITASQLDYENFKFYIQHGAAAYCNSETASGQKITCSDNGCKGVEANNAIIVASFVGKGTGIGGYVSTDNVRKEIVLSIRGSSNIRNWLTNVDFGQSSCSYVRDCGVHTGFRNAWDEIAQRARDAVAKARTMNPSYKVIATGHSLGGAVATLGAADLRSKGTAVDIFTFGAPRVGNAELSAFITAQAGGEFRVTHGRDPVPRLPPIVFGYRHTSPEYWLAGGASTKTDYTVNDIKVCEGAANLACNGGTLGLDIIAHLRYFQDTDACTAGGISWKRGDKAKRDEIPKRQEGMTDEELEQKLNDYVAMDKEYVESNKM